(19) United States
(12) Patent Application Publication
(10) Pub. No.: US 2012/0219274 A1
(43) Pub. Date: Aug. 30, 2012

(54) UFO shaped vaporizer

(76) Inventors: Mark Christopher Curran, JR., Fairfax, CA (US); Robert D. Schmid, Corte Madera, CA (US)

(21) Appl. No.: 12/932,448

(22) Filed: Feb. 28, 2011

(51) Int. Cl. A61L 9/03

(52) U.S. Cl. 392/386

(57) ABSTRACT

A UFO shaped assembly comprised of a top housing and a bottom housing. A vaporizer, comprised of an elongate metal heating element surrounded by a glass cylinder and a metal tube, is retained by a support plate within the housing. The the edge of the metal tube protrudes slightly above the surface through an aperture in the top housing. A rheostat controls the heat output of the heating element. An optional light transmitting disk and attached LEDs can be sandwiched between the top housing and the bottom housing thereby increasing the UFO effect. The vaporizer is powered by a power cord and plugged into a standard wall socket. The top housing may be removed from the bottom housing and replaced with other themed housings such as a sports car or a dragon.

### CROSS REFERENCE TO RELATED APPLICATIONS

Not Applicable

### STATEMENT REGARDING FEDERALLY SPONSORED RESEARCH OR DEVELOPMENT

Not Applicable

### DESCRIPTION OF ATTACHED APPENDIX

Not Applicable

### BACKGROUND OF THE INVENTION

This invention relates generally to the field of plant matter smoking devices and more specifically to a UFO shaped vaporizer.

Vaporizer assemblies have been used to help people smoke plant matter without inhaling the harmful tars and partials associated with burning plant matter. A vaporizer assembly heats the plant matter sufficiently do allow certain chemical compounds to be released without actually burning the plant matter.

To date, vaporizer assemblies used for smoking plant matter have been housed in conventional box-like housings and have no means for adjusting the temperature of the heating element contained within the vaporizer assembly.

There is a deficiency in the prior technology in that the conventional box-like shape normally associated with plant matter vaporizers may call unwanted attention to the intended use of the vaporizer assembly. Additionally, existing vaporizers do not have heat adjustment capability.

### BRIEF SUMMARY OF THE INVENTION

The primary object of the invention is to provide a vaporizer type plant matter smoking device that is concealed within a UFO shaped housing so as not to be associated with its actual purpose when not in use.

Another object of the invention is to provide a vaporizer whose outer UFO shaped shell can be removed and replaced with other novel housings including vehicle shapes and animal shapes.

Another object of the invention is to provide a vaporizer that includes a flashing light disk which produces a unique lighting effect around the perimeter of the vaporizer's housing.

A further object of the invention is to provide a vaporizer that allows the user to adjust the heat level of the vaporizing heating element.

Yet another object of the invention is to provide a vaporizer that uses a heating element with a swirling fin pattern to help improve air flow during use.

Still yet another object of the invention is to provide a vaporizer that includes a plant material retaining assembly that is easy to install and remove.

Other objects and advantages of the present invention will become apparent from the following descriptions, taken in connection with the accompanying drawings, wherein, by way of illustration and example, an embodiment of the present invention is disclosed.

In accordance with a preferred embodiment of the invention, there is disclosed a UFO shaped vaporizer comprising: an elongate metal heating element, a glass cylindrical surround member, a glass surround member retaining bracket, a metallic tubular surround member, a top housing, a bottom housing, a rheostat device, an rheostat adjustment knob, a support panel, a power cord and plug assembly, an on off switch, a tubular glass plant matter containing assembly, a flexible tube member, a mouthpiece member, said top and bottom housing forming a UFO shape, said elongate metal heating element terminating at one end in a circular flange, said heating element flange fixedly attached to said support panel, said glass cylindrical surround member surrounding said elongate heating element, said glass cylindrical surround member terminating at one end in a circular flange, said glass cylinder flange retained to said support panel by said glass surround member retaining bracket, said metallic tubular surround member surrounding said glass cylinder and attached to said support panel, said elongate metal heating element capable of temperature adjustment by said rheostat device, said rheostat adjustable by said adjustment knob, said top housing attached to said bottom housing by said housing screws, said top housing having an aperture sized to allow the tip of said metallic tubular surround member to be exposed, said top housing removable and replaceable from said bottom housing by loosening said housing screws, said tubular glass plant containing assembly including a first glass tube, a second glass tube, a first screen, a second screen and an outer retaining ring, said outer retaining ring fixedly attached to said first glass tube and slidably joining said first glass tube to said second glass tube, said first glass tube retaining a metal screen disk at one end, said second glass tube retaining a metal screen disk at one end, the space between said screen disks capable of removably retaining plant matter, one end of said tubular glass plant matter containing assembly slidably and removably attached to the outside of said glass surround member, the opposite end of said glass plant containing assembly attached to said flexible tube member, the opposite end of said flexible tube member attached to said mouthpiece member, said power cord and plug assembly attached to said rheostat and said elongate metal heating element, and said on off switch mounted in line on said power cord.

### BRIEF DESCRIPTION OF THE DRAWINGS

The drawings constitute a part of this specification and include exemplary embodiments to the invention, which may be embodied in various forms. It is to be understood that in some instances various aspects of the invention may be shown exaggerated or enlarged to facilitate an understanding of the invention.

FIG. 1 is a perspective view of the invention.

FIG. 2 is a a perspective view of the invention with a conceiting cover in place.

FIG. 3 is a side view of the invention.

FIG. 4 is a bottom view of the invention.

FIG. 5 is an exploded view of the plant matter holding portion of the invention.

FIG. 6 is a section view of the vaporizer elements of the invention.

FIG. 7 is a rear view of the invention.

FIG. 8 is a perspective view of the invention with the top housing removed.

FIG. 9 is a plan view of the light transmitting ring.

FIG. 10 is a side section view of the invention.

FIG. 11 is a partial side view of the heating element.

FIG. 12 is a perspective view of an alternate embodiment of the top housing.

### DETAILED DESCRIPTION OF THE PREFERRED EMBODIMENTS

Detailed descriptions of the preferred embodiment are provided herein. It is to be understood, however, that the present invention may be embodied in various forms. Therefore, specific details disclosed herein are not to be interpreted as limiting, but rather as a basis for the claims and as a representative basis for teaching one skilled in the art to employ the present invention in virtually any appropriately detailed system, structure or manner.

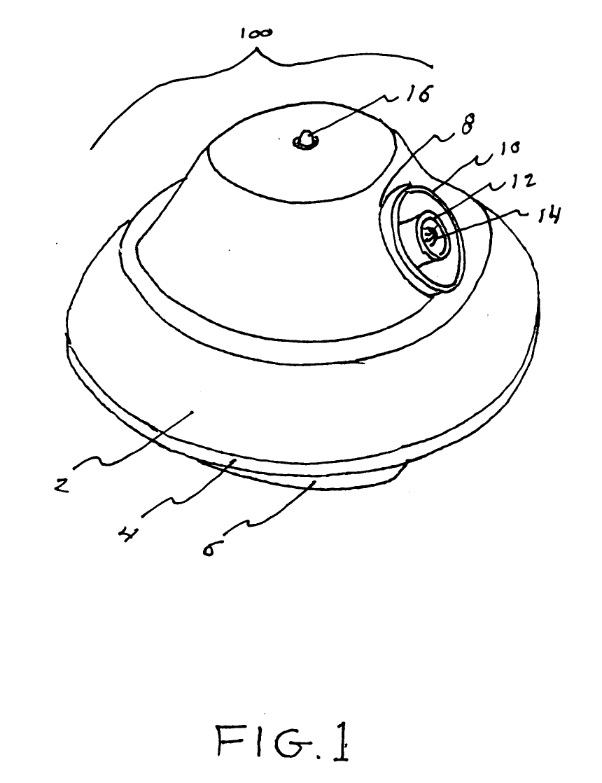

Referring now to FIG. 1 we see a perspective view of the UFO shaped vaporizer 100 of the present invention. UFO stands for unidentified flying object. The flying saucer shape is commonly thought of when visualizing UFOs.

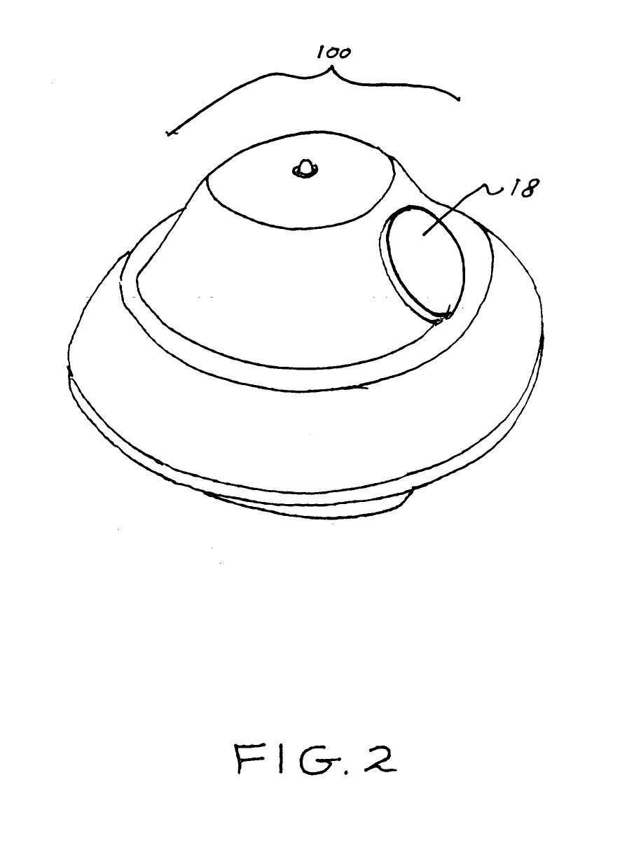

The unique shape, formed of top housing 2 and bottom housing 6, masks the fact that a vaporizer mechanism is located within the housing. This feature is unique and important when a person wants to have a vaporizer unit in plain sight but does not want to draw attention to its ultimate use, which is for smoking plant matter. The UFO effect is further enhanced by optional light emitting ring 4. An additional optional light 16 is located at the top center of housing 2. Metal tube 10, glass tube 12 and heating element 14 all protrude slightly from aperture 8. These elements are part of the vaporizing process, but also can be considered part of the UFO design. For further concealment of the intended use of the device 100, a cover plate 18 can be removably attached to the metal tube 10 as shown in FIG. 2

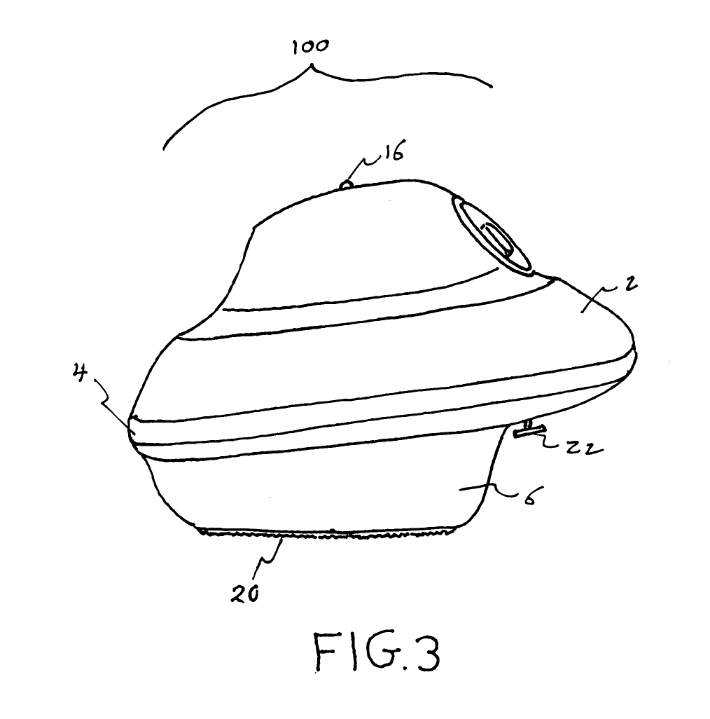

FIG. 3 is a side view of the present invention 100. A rubber-like non slip bottom panel 20 can be seen as well as heater adjustment knob 22 which will be described in detail below. The UFO shape is positioned at an angle which represents the UFO getting ready to take off.

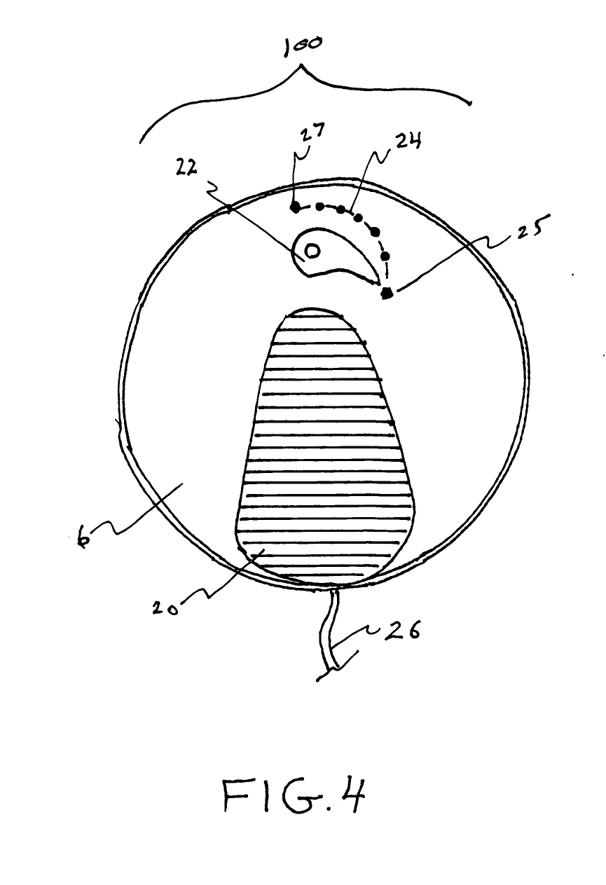

FIG. 4 is a bottom view of the invention 100. Non slip panel 20 can be clearly seen as well as heater adjustment knob 22 and adjustment setting indicator 24. The highest adjustment setting 25 makes the heater element operate at approximately 400 degrees F. The lowest setting 27 makes the heater element operate at approximately 350 degrees F. The setting can be optimized by the user to accommodate the type of plant material being smoked and intensity of the inward draw breath of the user.

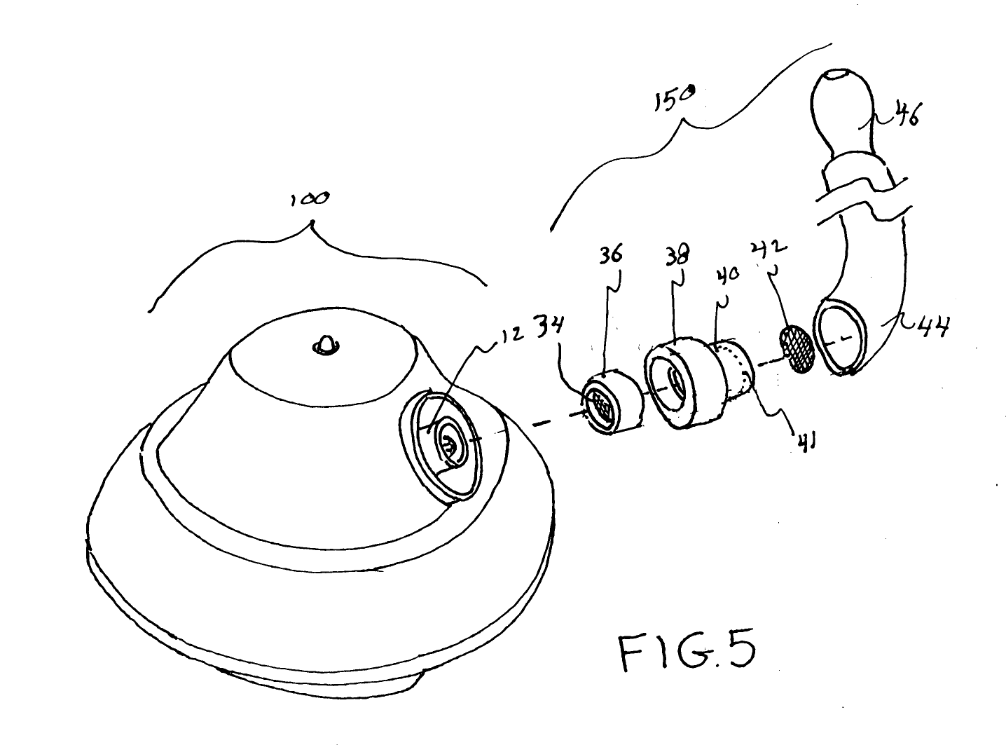

FIG. 5 shows an exploded view of the components of the plant matter holding assembly 150 and its relationship to main vaporizer unit 100. First glass cylinder 36 can slip onto glass tube 12 of the vaporizer unit 100. A sleeve 38 is attached to second glass cylinder 40. The sleeve 38 can slip over first glass cylinder 36 in an air tight manner. A first screen 34 is fixed inside glass cylinder 36. A second screen 42 is placed inside glass cylinder 40 at dotted line 41. A flexible tube 44 fits over glass cylinder 40 at one side and over mouthpiece 46 at the opposite side.

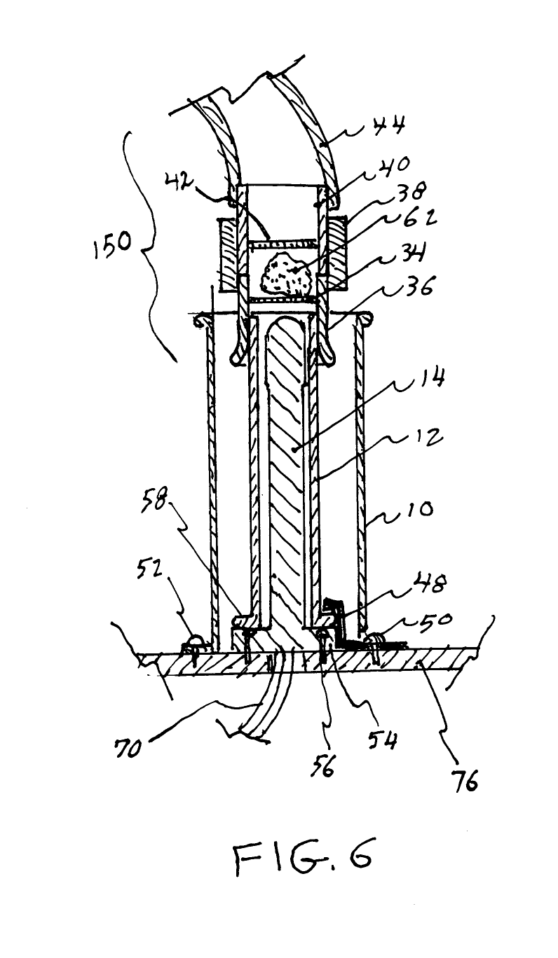

The section view shown in FIG. 6 bisects the heater assembly and plant matter retaining assembly 150. Heater element 14 is powered by 120 volt AC power coming from power cord 70. A flange 54 located at the bottom of the heating element 14 is screwed 56 to support plate 76. A glass tube 12 surrounds heating element 14 thereby directing heat up to plant matter 62. An outer metal tube 10 further directs heat upward and protects the rest of the outer housing top 2 from excessive heat contact. Glass tube 12 has a flange 58 at its bottom most area. the flange 58 is held in place by tube holding bracket 48 which is made of metal and secured to support plate 76 by screw 50. Metal tube 10 is held to support plate 76 by tab and screw 52. The first glass cylinder 36 is slightly flared allowing it to slip easily onto glass tube 12. Sleeve 38 can be seen holding glass cylinder 36 and glass cylinder 40 together. Screens 34, 42 retain plant matter 62.

To insert plant matter 62 the user separates cylinder 36 from cylinder 40, inserts the material 62 and then slides the two cylinders 36, 40 together again. Flexible tube 44 can be seen attached to one end of cylinder 40. When the user draws breath inward from mouthpiece 46, heated air generated by heating element 14 is caused to be drawn through screen 34, then heating plant matter 62, and then vaporized plant matter is drawn through screen 42 and up tube 44 to mouthpiece 46 and into the user's mouth and lungs.

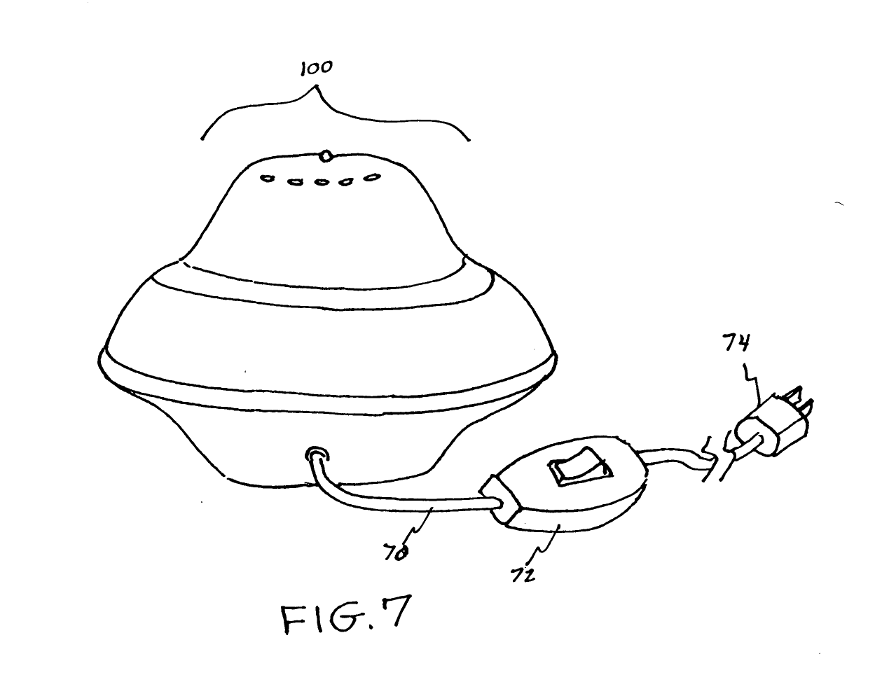

FIG. 7 shows a rear view of the invention 100. Power cord 70 and wall plug 74 are clearly seen as is power on-off switch 72.

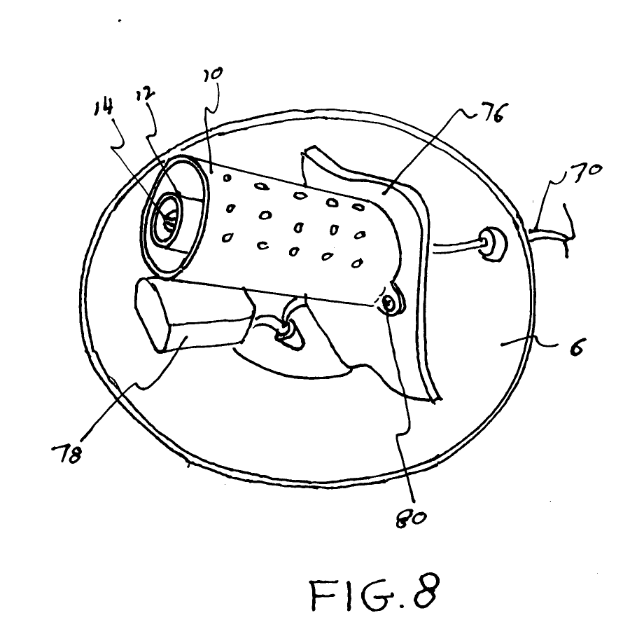

FIG. 8 is a perspective view showing the base housing 6 with support plate 76 in place and supporting metal tube 10, glass tube 12 and heater element 14. Rheostat 78 helps adjust the heat output of heater element 14. The rheostat 78 is adjusted by knob 22 described in FIG. 4.

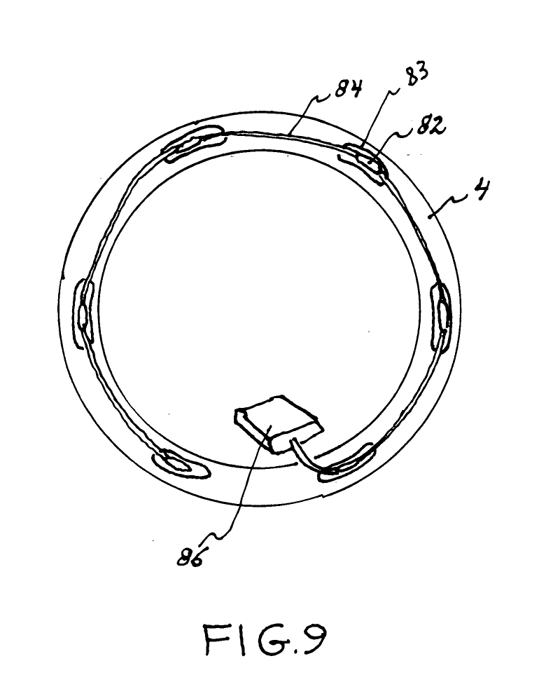

FIG. 9 is a plan view of the light transmitting disk 4. LEDs 82 are inserted into apertures 83 and wired together through electrical cable 84. The LEDs 82 are powered by AC to DC transformer 86 which is in turned powered by a standard 110 VAC power source. Light is transferred to the perimeter of the disk 4 causing a flashing illumination that enhances the UFO effect. It should be noted that a version of the invention 100 can be made without the light disk 4 and still be within the spirit and scope of the present invention.

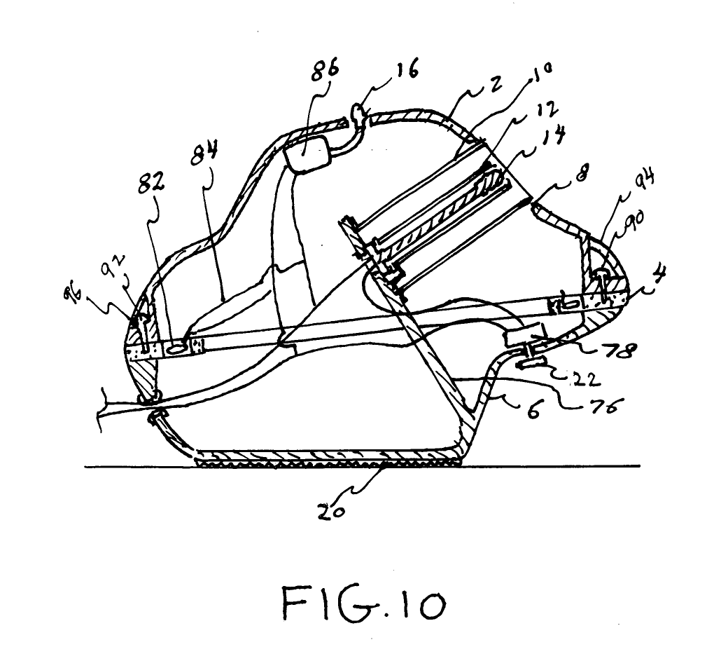

FIG. 10 is a side section view that bisects the invention 100. All internal components can be clearly seen. AC to DC transformer 86 also powers top LED 16. Light transmitting disk 4 can be clearly seen as well as the location of LEDs 82. Top housing 2 is held in place by screws 90, 92 and concealed by cover plates 94, 96.

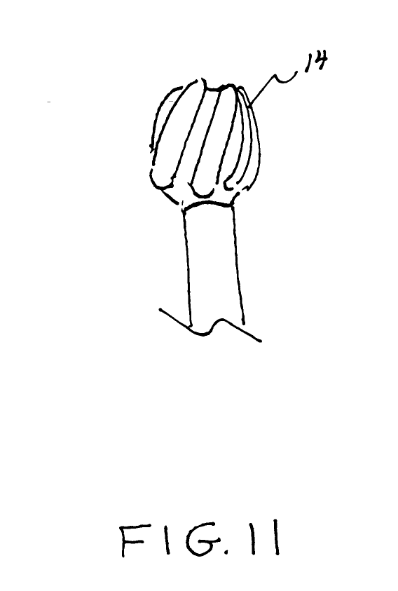

FIG. 11 shows a partial side view of heating element 14. The tip of the element has a swirl design that helps direct heated air in a swirling pattern thereby improving the ability of the drawn air to fully vaporize the plant matter 62.

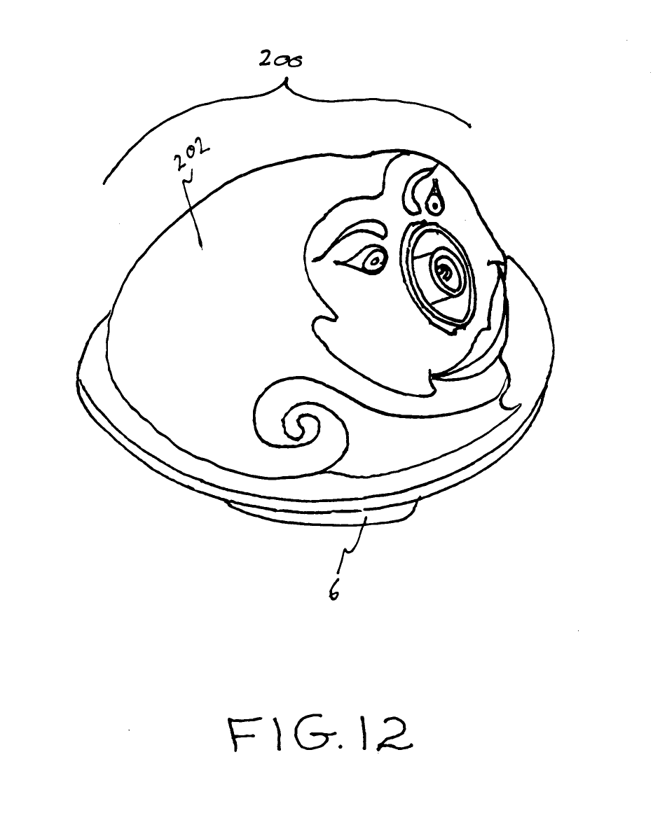

FIG. 12 shows an alternate embodiment of the invention 200 where a fantasy animal shape 202 has replaced the UFO shape of the first embodiment 100. Other decorative top housings may be substituted such as a race car or a dragon.

While the invention has been described in connection with a preferred embodiment, it is not intended to limit the scope of the invention to the particular form set forth, but on the contrary, it is intended to cover such alternatives, modifications, and equivalents as may be included within the spirit and scope of the invention as defined by the appended claims.

What is claimed is:

1. UFO shaped vaporizer comprising:
an elongate metal heating element;
a glass cylindrical surround member;
a glass surround member retaining bracket;
a metallic tubular surround member;
a top housing;
a bottom housing;
a rheostat device;
an adjustment knob;
a support panel;
a power cord and plug assembly;
an on off switch;
a tubular glass plant matter containing assembly;
a flexible tube member;
a mouthpiece member;
said top and bottom housing forming a UFO shape;
said support panel fixedly attached to the inside of said bottom housing;
said elongate metal heating element terminating at one end in a circular flange;
said heating element flange fixedly attached to said support panel;
said glass cylindrical surround member surrounding said elongate heating element;
said glass cylindrical surround member terminating at one end in a circular flange;
said glass cylinder flange retained to said support panel by said glass surround member retaining bracket;
said metallic tubular surround member surrounding said glass cylinder and attached to said support panel;
said elongate metal heating element capable of temperature adjustment by said rheostat device;
said rheostat adjustable by said adjustment knob;
said top housing attached to said bottom housing by said housing screws;
said top housing having an aperture sized to allow the tip of said metallic tubular surround member to be exposed;
said top housing removable and replaceable from said bottom housing by loosening said housing screws;
said tubular glass plant containing assembly including a first glass tube, a second glass tube, a first screen, a second screen and an outer retaining ring;
said outer retaining ring fixedly attached to said second glass tube and slidably joining said first glass tube to said second glass tube;
said first glass tube retaining a metal screen disk at one end;
said second glass tube retaining a metal screen disk at one end;
the space between said screen disks capable of removably retaining plant matter;
one end of said tubular glass plant matter containing assembly slidably and removably attached to the outside of said glass cylinder member;
the opposite end of said glass plant containing assembly attached to said flexible tube member;
the opposite end of said flexible tube member attached to attached at one end to said mouthpiece member;
said power cord and plug assembly attached to said rheostat and said elongate metal heating element; and
said on off switch mounted in line on said power cord.

2. UFO shaped vaporizer as claimed in claim 1 further comprising a light conducting disk mounted between said top housing and said bottom housing;
said disk having a plurality of apertures which each containing an LED light source;
said LED lights powered by an AC to DC transformer which is in turned powered by said power cord and plug assembly

3. UFO shaped vaporizer as claimed in claim 1 wherein said top housing is removable and can be replaced with other housings that are designed to look like other items such as a vehicle an animal;
each said alternate top housing having an aperture that allows said the outer tip of metallic tubular surround member to be exposed.

4. UFO shaped vaporizer as claimed in claim 1 further comprising an additional LED light that can be seen through a central aperture in the said top housing.

5. UFO shaped vaporizer as claimed in claim 1 further comprising a removable and replaceable closure lid for said metallic tubular surround member thereby conceiting the items located within said metal tubular surround member.
